(19) United States
(12) Patent Application Publication
(10) Pub. No.: US 2010/0093107 A1
(43) Pub. Date: Apr. 15, 2010

(54) POLYMER-PROTEIN SUBSTRATES FOR IMMUNOSORBENT FLUORESCENCE ASSAYS

(75) Inventors: Lisa-Jo Clarizia, Albuquerque, NM (US); Melisenda McDonald, Westford, MA (US); Joey Mead, Carlisle, MA (US)

Correspondence Address: WOLF GREENFIELD & SACKS, P.C., 600 ATLANTIC AVENUE, BOSTON, MA, 02210-2206, US

(73) Assignee: University of Massachusetts, Boston, MA (US)

(21) Appl. No.: 12/448,238

(22) PCT Filed: Dec. 13, 2007
(86) PCT No.: PCT/US2007/025631

§ 371 Date: Dec. 15, 2009

Related U.S. Application Data
(60) Provisional application No. 60/874,807, filed on Dec. 14, 2006.

(51) Int. Cl. G01N 33/53; G01N 33/00; B05D 3/00

(52) U.S. Cl. 436/501; 422/69; 427/2.13

(57) ABSTRACT

The present invention relates to antigen-capture substrates useful for orienting capture antibodies for immunosorbent antigen determination.

### FIELD OF THE INVENTION

The present invention relates to the field of antigen determination.

### BACKGROUND OF THE INVENTION

ELISA (Enzyme-Linked Immunosorbent Assay) comprises the determination of an antigen in a sample. One form of ELISA, the sandwich ELISA, comprises the determination of an antigen by binding of the antigen to two antibodies, a primary antibody for capture of the antigen and a secondary antibody for detection of the antigen. A primary antibody is immobilized by adsorption to a solid surface. The antigen is subsequently bound to (captured by) the immobilized primary antibody. The next step of the sandwich ELISA assay comprises binding of the secondary antibody to the antigen. The secondary antibody will allow for the detection of the bound antigen. To allow for detection, the secondary antibody is coupled to a detectable label or to an enzyme that will facilitate detection. Alternatively, a third antibody coupled to a detectable label can bind to the secondary antibody to facilitate detection.

The primary antibody can be adsorbed to a solid surface via passive, non-covalent binding (generally electrostatic and hydrophobic interactions). The solid surface is generally a plastic, like polystyrene, which is favored because of its protein-binding capacity, optical properties, relative low cost, and ease of manufacture. The adsorption of an antibody to the solid surface is non-specific in nature, i.e., either the Fc region or the Fab domains of the antibody may bind to the solid surface during the coating step. However, the antibody can bind an antigen only through the Fab domains. The detection capacity of an ELISA can therefore be improved if the adsorbed antibodies have the correct orientation, i.e., were to be coupled or adsorbed to the solid surface through the Fc region, leaving the Fab domains available for antigen binding.

### SUMMARY OF THE INVENTION

The invention provides compositions and methods that improve the orientation of antibodies, as well as other Fc-containing proteins and polypeptides, on a surface so as to enhance interaction between non-Fc portions of the antibodies or other Fc-containing proteins and polypeptides with a sample. The compositions and methods of the invention are useful in a number of in vitro molecular interaction/detection assays, including but not limited to enzyme-linked immunosorbent assay (ELISA), fluorescent-linked immunosorbent assay (FLISA), and surface plasmon resonance.

In one aspect the invention is an antigen-capture substrate comprising a solid surface coated with a polymer and an antibody-binding protein coupled to the polymer, wherein the antibody-binding protein can bind an Fc region of an antibody. In various embodiments the polymer is poly-methyl methacrylate (PMMA), poly-acrylic acid, poly-styrenesulfonate/poly-2,3-dihydrothieno(3,4-b)-dioxin, poly-aniline, or poly-styrene-co-methyl methacrylate. In various embodiments the polymer is polyamide, polyethylene oxide, polystyrene, etc. In one embodiment according to this aspect of the invention the antigen-capture substrate further comprises an antibody bound to the antibody-binding protein.

In another aspect the invention is a method for producing an antigen-capture substrate. The method according to this aspect of the invention includes the steps of (a) coating a solid surface with a polymer, and (b) coupling an antibody-binding protein to the polymer, wherein the antibody-binding protein can bind an Fc region of an antibody. In various embodiments the polymer is poly-methyl methacrylate (PMMA), poly-acrylic acid, poly-styrenesulfonate/poly-2,3-dihydrothieno(3,4-b)-dioxin, poly-aniline, or poly-styrene-co-methyl methacrylate. In one embodiment according to this aspect of the invention the method further includes the step of coupling an antibody to the antibody-binding protein.

In yet another aspect the invention is a method for determining presence of an antigen in a sample. The method according to this aspect of the invention includes the steps of (a) contacting the antigen-capture substrate of the invention with the sample, wherein an antibody is coupled to the antibody-binding protein and the antibody binds specifically to the antigen when the antigen is present; and (b) determining if the antibody bound the antigen, wherein if the antibody bound the antigen, the antigen is determined to be present in the sample. In one embodiment the method according to this aspect of the invention further includes the step of (c) determining the amount of the antigen bound to the antibody, wherein the amount of the antigen bound to the antibody correlates with the amount of antigen in the sample. In one embodiment according to this aspect of the invention either or both determining steps comprise an absorbance measurement. In one embodiment according to this aspect of the invention either or both determining steps comprise a fluorescence measurement.

Each of the foregoing aspects and embodiments of the invention can embrace the following embodiments.

In one embodiment the polymer is poly-methyl methacrylate (PMMA).

In one embodiment the antibody-binding protein is protein G.

In one embodiment the antibody-binding protein is a truncated protein G.

In one embodiment the antibody-binding protein is protein G′ expressed in Streptococci sp. G148.

In one embodiment the antibody-binding protein is protein A.

In one embodiment the antibody-binding protein is protein L.

In one embodiment the antibody is an IgG.

In one embodiment the IgG is a human IgG.

In one embodiment the solid surface is polystyrene.

In one embodiment the solid surface is a multi-well plate.

In one embodiment the solid surface is glass.

In one embodiment the solid surface is a slide.

In one embodiment the antibody-binding protein is non-covalently coupled to the polymer.

In one embodiment the antibody-binding protein is coupled at a concentration of 0.01, 0.1, 1, 2, 5, 10, 50 or 100 microgram/ml.

In one embodiment the antibody-binding protein is coupled at a concentration of 1 microgram/ml.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF THE DRAWINGS

FIG. 1 shows a method for solid casting, a) mechanical spreading, b) solvent evaporation and crystallization (or solidification).

FIG. 2 is a graph depicting the Fab and Fc response for polystyrene (PS), poly-methyl methacrylate (PMMA), poly-acrylic acid (PA), poly(styrenesulfonate)/poly(2,3-dihydrothieno(3,4-b)-dioxin (PPD), and poly-aniline (PANI). G refers to the presence of protein G′.

FIG. 3 is a graph depicting the relative fluorescence for polystyrene, polystyrene/PMMA, and PMMA. G and no G refer to the presence or absence, respectively, of protein G′.

FIG. 4 is a graph depicting the relative fluorescence for PMMA, ultra-high-binding (UHB) polystyrene, high-binding (HB) polystyrene, and medium-binding (MB) polystyrene.

FIG. 5 is a graph depicting the Fab response as measured by relative fluorescence for PMMA, UHB, HB and MB styrene with protein G′ coating.

FIG. 6 is a graph depicting the Fc response as measured by relative fluorescence for PMMA, UHB, HB and MB styrene with protein G′ coating.

FIG. 7 is a graph depicting the relative fluorescence for polystyrene control, PMMA, and polystyrene film.

FIG. 8 is a graph depicting the relative fluorescence for polystyrene and PMMA as measured by fluorescein isothiocyanate (FITC) and tetramethylrhodamine isothiocyanate (TRITC).

FIG. 9 is a graph depicting an anti-Fab-FITC intensity plot for PMMA and polystyrene.

FIG. 10 shows an anti-Fab-FITC intensity overview photograph for PMMA and polystyrene.

### DETAILED DESCRIPTION

In some embodiments the invention provides methods and substrates for optimized protein orientation. In one embodiment of the invention proteins are oriented through binding to an antigen-capture substrate. The antigen-capture substrate of the invention comprises a polymer and an antibody-binding protein coupled to the polymer. The antibody-binding proteins of the antigen-capture substrate can bind the Fc region of antibodies. The invention embraces compositions and methods for the orientation of any protein that comprises an Fc region of an antibody. Thus any protein or polypeptide with an Fc region, including antibodies and Fc fusion proteins, can be oriented by the antigen-capture substrate of the invention.

The antigen-capture substrate of the invention comprises a solid surface coated with, or otherwise bearing, a polymer to which is coupled an antibody-binding protein. In some embodiments of the invention the solid surface is coated with the polymer. In some embodiments the polymer has an amphipathic surface. In some embodiments the polymer is adsorbed to a solid surface. In some embodiments the polymer is poly-methyl methacrylate (PMMA). In some embodiments the antibody-binding protein is protein G or an antibody-binding fragment of protein G. In some embodiments the polymer is PMMA and the antibody-binding protein is protein G or an antibody-binding fragment of protein G.

Many applications, including ELISA, require antibodies to be immobilized and able to bind antigens. Antibodies bind antigens through their Fab domains. Thus, immobilized antibodies that have their Fab domains available for binding to antigen are preferred over immobilized antibodies that do not have their Fab domains available for binding to antigen. Similarly, immobilized antibodies that have their Fab domains more available for binding to antigen are preferred over immobilized antibodies that have their Fab domains less available for binding to antigen. Binding of the Fc region of an antibody to the antigen-capture substrate leaves the Fab domains more available for binding to the antigen. The invention embraces antibodies bound to the antigen-capture substrate thought their Fc region. In some embodiments the antigen-capture substrate of the invention comprises the bound antibody.

In one embodiment the invention provides antigen-capture substrates and methods for immunosorbent assays, including ELISAs. The antigen-capture substrate of the invention, bearing an antibody specific for a particular antigen, allows for improved capture of the particular antigen from a sample, thereby allowing for the determination of the presence and/or quantity of the antigen in the sample. In some embodiments binding of antigen to an antigen-specific antibody, which antibody is bound to and oriented by the antigen-capture substrate of the invention, allows for the determination of the amount of antigen present in a sample.

In immunosorbent assays, including ELISA, the primary antibody can be adsorbed to a solid surface via passive, non-covalent binding, including electrostatic and hydrophobic interactions (16). The solid surface can be a plastic, an organic solid phase substrate such as polysaccharide-derived beads, or an inorganic solid phase substrate such as silica glass or metal. The nature of the solid surface determines its antibody adsorption properties (17). Plastics, most notably polystyrene, are often used as solid surfaces for ELISA due to their protein-binding capacity, optical properties, low cost and ease of manufacture (18). Regardless of identity of the solid surface, it is preferred that the Fc region of the antibody adheres to the solid surface, leaving the Fab domain free to bind antigen (21). However, non-oriented adsorption results in a random non-ideal orientation of a subset of antibodies. Adsorption of an antibody to the solid surface is non-specific in nature; either the Fc or the Fab domain may bind to the plate during the coating step (22). To the extent that an Fab domain is bound to the solid surface, it is unavailable for binding the antigen. In some studies as little as 20% of primary antibodies oriented with the Fab domain are available for antigen binding upon adsorption to a solid surface (23). For this reason, the primary antibody is typically applied in excess to ensure enough properly oriented antibodies for antigen capture. Another disadvantage of non-oriented adsorption is the possibility of auto-binding between the antibodies. This binding of antibodies to each other also results in fewer Fab domains available for antigen binding.

Several strategies have been employed to try to orient antibodies correctly, for example by including chemical modification of the Fc region, by adding thiol (26), or by conjugating the Fc region of the antibody to a chemical linker, such as gold (27), which is then bound to a specially-treated solid surface (28). However, these methods often resulted in loss of biological efficacy of the antibodies.

As an alternative to chemical methods, antibodies can be oriented using antibody-binding proteins. Several bacterial proteins, including protein G, protein A, and protein L, are known to bind to the Fc region of antibodies (31). As with antibodies, and any other protein, the adsorption of an antibody-binding protein to a solid surface can arise from non-covalent binding, principally electrostatic interactions (40). Proteins are amphipathic in nature and may bind either to hydrophilic or hydrophobic surfaces. Therefore, the characteristics of a given solid surface will play a large role in the attachment of the antibody-binding protein to the solid surface. The antigen-capture substrate of the invention provides for optimized binding of antibody-binding protein to a solid surface through a polymer. In one embodiment the solid surface is coated with a polymer and the antigen-binding protein is coupled to the polymer.

### Antigen-Capture Substrate

An antigen-capture substrate comprises a solid surface that is either made of a polymer or coated with a polymer, wherein the polymer is coupled to an antibody-binding protein as described herein. Any combination of antibody-binding proteins of the invention coupled to polymers of the invention is embraced by the invention. In some embodiments the antigen-capture substrate comprises the polymer PMMA coupled to protein G. In some embodiments the antigen-capture substrate comprises the polymer PMMA coupled to protein G′. The combination of polymer, e.g., PMMA and antibody-binding protein, e.g., protein G, facilitates the correct orientation of proteins with an Fc region. Similarly, the combination of polymer, e.g., PMMA and antibody-binding protein, e.g., protein G′, facilitates the correct orientation of proteins with an Fc region.

In some embodiments the antigen-capture substrate further comprises a protein having an Fc region, wherein the protein having the Fc region is coupled to the antibody-binding protein. In some embodiments the antigen-capture substrate further comprises an antibody, wherein the antibody is coupled to the antibody-binding protein.

### Solid Surface

Solid surfaces embraced by the invention include but are not limited to solid materials at room temperature formed from any suitable material such as polymers, including poly-styrene, poly-methyl methacrylate (PMMA), poly-acrylic acid, poly-aniline, poly-styrene-co-methyl methacrylate, and poly-styrenesulfonate/poly-2,3-dihydrothieno(3,4-b)-dioxin, and copolymers thereof, glass, starch, and metal. In some embodiments the solid surface is inert to common organic solvents like acetone and toluene, such that solutions of polymer or of polymer components prepared in such solvents can be effectively used in combination with the solid surface.

In some embodiments of the invention the solid surface which is coated by the polymer is polystyrene. Polystyrene, an aromatic, thermoplastic polymer (35), is sturdy, inexpensive, optically clear, amenable to surface treatments (such as oxygen plasma deposition), and can be sterilized by gamma irradiation or ethylene oxide gas.

Embodiments of the shape of the solid surface include, but are not limited to multiwell plates, including 6-, 8-, 12-, 24-, 36-, 48-, 72-, 96-, and 364-well plates, slides, and beads. In certain embodiments the solid surface is provided as a multiwell plate. In one embodiment the solid surface is not a bead. In certain embodiments the polymer is coated onto the solid surface such that the polymer, and the antibody-binding protein coupled to it, will come into contact with or otherwise face a sample. In one embodiment the plates, and similarly other solid surfaces according to the invention, can be directly molded from the polymer.

### Polymers

The invention embraces any polymer that can couple to an antibody-binding protein. In some embodiments the polymer allows for the orientation of the binding sites of the antibody-binding protein away from the solid surface, and thus available for binding. In some embodiments the polymer provides an amphipathic surface for protein adsorption. Polymers useful according to the invention include, but are not limited to poly-methyl methacrylate (PMMA), poly-acrylic acid (PAA), poly-aniline (PANI), poly-styrene-co-methyl methacrylate, poly-styrene (PS), poly-styrenesulfonate/poly-2,3-dihydrothieno(3,4-b)-dioxin, polyamide, polyethylene oxide, and copolymers thereof.

Polymers can be coated onto the solid surface using a variety of techniques. In some embodiments the polymer is coated onto the substrate through solution casting. Solution casting is a technique routine in the art and comprises solubilizing the polymer into an appropriate solvent and dispersing the polymer. Once the polymer is dispersed across the solid surface the solvent is evaporated or is allowed to evaporate, resulting in a layer of polymer coating over the solid surface. The evaporation may result in crystallization or solidification of polymer, depending on the nature of the polymer. The polymer surface can also be produced by a variety of other polymer processing techniques, such as injection molding, thermoforming, extrusion, etc.

In some embodiments of the invention the polymer is actively coupled to the solid surface. Active coupling includes an increase in temperature, exposure to light, pressure or chemical reactions. The interaction between the polymer and the solid surface can be of any nature. The invention embraces any kind of interaction between the polymer and the solid surface including but not limited to, hydrophobic, hydrophilic, van der Waals, ionic, and covalent, and any combination thereof.

In some embodiments of the invention a precursor of the polymer is dispersed onto the solid surface, after which the building blocks can subsequently be polymerized in situ. Precursors include the monomeric building blocks of the polymer, i.e., the repeating unit of the polymer, and any combination of chemical components that can result in the formation of a polymer. In some embodiments the components are added sequentially. In some embodiments the precursor components are coupled to the solid surface before polymerization is induced.

In some embodiments of the invention the polymer and the solid surface are the same material. In such embodiments the solid surface need not be coated with the polymer. Alternatively, even if both the solid surface and the polymer are the same material, in some embodiments the polymer is coated and dispersed onto the solid surface as described above. In some embodiments both the solid substrate and the polymer are PMMA.

### Antibody-Binding Protein

An antibody-binding protein is any protein that can bind an Fc-containing protein or polypeptide, Fc-containing antibody, or Fc-containing fragment of an Fc-containing antibody. In some embodiments antibody-binding proteins bind the Fc region of antibodies. In some embodiments the antibody-binding protein of the invention is a bacterial antibody-binding protein. Bacterial antibody-binding proteins are well known in the art and include but are not limited to protein G, protein L, protein A, derivatives thereof (including but not limited to protein G′), and combinations thereof. Bacterial antibody-binding proteins are commercially available for instance from Pierce Biotechnology (Rockford, Ill.). Each bacterial antibody-binding protein has a specific affinity for each class (i.e., isotype) of antibody and each species from which the antibody is derived. For instance, protein G strongly binds both human and goat IgG, whereas protein A strongly binds human IgG but only weakly binds goat IgG. The current invention embraces all antibody-binding proteins regardless of their specific binding properties.

In some embodiments the antibody-binding protein is protein G, which is derived from the Streptococci sp. bacteria. The native protein G consists of six subunits (A1, A2, B1, B2, C1, C2). The A1 and A2 subunits bind the Fab domain of antibodies. The B1 and B2 subunits selectively bind the Fc region of antibodies belonging to the immunoglobulin subclass IgG, with each subunit having three immunoglobulin binding sites. The C1 and C2 subunits bind albumin.

The invention embraces variants and truncated versions of protein G that minimally comprise the subunits that bind to the Fc region of antibodies. One commercially available truncated version of Protein G is Protein G′, which is a recombinant form of Protein G expressed in the G148 strain of Streptococci sp. bacteria and expressed in E. coli (33). This recombinant protein lacks albumin- and Fab-binding subunits (A1, A2 and C1, C2, respectively) of protein G. However, protein G′ does encompass the two subunits (B1 and B2) that bind the Fc region of antibodies exclusively.

The antigen-capture substrate comprises antibody-binding proteins coupled to the polymer. The invention embraces any method of coupling the antibody-binding protein to the polymer including an increase in temperature, exposure to light, pressure or chemical reactions. The coupling between the antibody-binding protein and the polymer can be of any nature including but not limited to hydrophobic, hydrophilic, van der Waals, ionic, covalent, and non-covalent interactions, and any combination thereof. In some embodiments the coupling between the antigen-binding protein and polymer is non-covalent.

### Antibody

The antigen-capture substrate of the invention can bind to any protein that includes an Fc region. Proteins with an Fc region include, but are not limited to, antibodies and Fc-fusion proteins. Any protein, antibody or antibody fragment comprising at least one Fc region is embraced by the invention. In some embodiments the antibody will comprise at least one Fc region and at least one Fab domain.

In one embodiment the Fc-containing antibody or protein is coupled to the antibody-binding protein of the antigen-capture substrate of the invention. The coupling between the Fc-containing antibody or protein and the antibody-binding protein of the antigen-capture substrate of the invention can be of any nature including but not limited to hydrophobic, hydrophilic, van der Waals, ionic, covalent, and non-covalent interactions, and any combination thereof. In some embodiments the coupling between the Fc-containing antibody or protein and the antigen-binding protein is non-covalent.

Each antibody specifically binds an antigen or group of antigens with similar structural properties. Antibody specificity is determined by the complementarity determining regions (CDRs). These CDRs are located near the amino terminal of each Fab domain and are about 7-22 amino acid residues in length. Antibodies are comprised of a heavy chain (50-70 kDa; 440-550 amino acid residues) of type α, γ, δ, ε or μ and a light chain (23-25 kDa; 220 amino acid residues) of type λ or κ. The heavy and light chains are linked together by interchain disulfide bonds and non-covalent interactions. The number and location of interchain disulfide bonds varies between and within antibody classes. In addition to interchain disulfide bonds, intrachain disulfide bonds and non-covalent interactions can occur within each heavy and light chain. Both heavy chains and light chains are comprised of a constant and variable region. The constant regions of the heavy chain (C_{H}) consist of 330-440 amino acid residues, with the light chain constant region (C_{L}) consisting of 110 amino acid residues. The variable region of the heavy chain (V_{H}) and light chain (V_{L}) consist of 110 amino acids residues each.

Antibodies are comprised of two globular regions, also referred to as domains, an Fc domain located at the carboxyl terminal of the molecule and two Fab domains located near the amino terminal. Thus, each antibody consists of one Fc domain and two antigen-binding Fab domains (also referred to as Fab arms). Each antibody Fab arm can bind a different antigen in principle. Antibodies that comprise two different Fab arms are referred to as multivalent antibodies.

In some embodiments the antibody is an IgG. In one embodiment the IgG is a human IgG. In some embodiments the antibody or antigen-binding fragment thereof is selected from the group consisting of IgG1, IgG2, IgG3, IgG4, IgM, IgA 1, IgA2, IgAsec, IgD, IgE or has immunoglobulin constant and/or variable domain of IgG1, IgG2, IgG3, IgG4, IgM, IgA1, IgA2, IgAsec, IgD or IgE. In some embodiments the antibody is a bispecific or multispecific antibody. In some embodiments the antibody is a recombinant antibody, a polyclonal antibody, a monoclonal antibody, a humanized antibody, a chimeric antibody, or any mixture of these. In some embodiments the antibody is a human antibody. In some embodiments, the antibody is a bispecific or multispecific antibody.

In some embodiments the antibody is a polyclonal antibody. The production of polyclonal antibodies is routine in the art. Polyclonal antibodies can be prepared by a variety of methods, including administering a protein, fragments of a protein, cells expressing the protein or fragments thereof and the like to an animal to induce polyclonal antibodies, which can subsequently be harvested from the serum of the animal.

In some embodiments the antibody is a monoclonal antibody. The production of monoclonal antibodies is well known in the art. Briefly, to produce monoclonal antibodies, typically a mammal such as a mouse is injected with a protein, fragments of a protein, cells expressing the protein or fragments thereof and the like. Subsequently, the spleen of the injected animal is removed and serves as a source of lymphocytes, some of which are producing antibody of the appropriate specificity. Spleen cells are then fused with a permanently growing myeloma partner cell, and the products of the fusion are plated into a number of tissue culture wells in the presence of a selective agent such as HAT. The wells are then screened to identify those containing cells making useful antibody, for example by ELISA. Cells from each well with a positive ELISA signal are then freshly plated. After a period of growth, these wells are again screened to identify antibody-producing cells. Several cloning procedures are carried out until over 90% of the wells contain single clones which are positive for antibody production. From this procedure a stable line of clones is established which produces the monoclonal antibody.

In some embodiments the antibodies are chimeric. Chimerization comprises replacing sequences or elements of a first species, e.g., human Fab domains, with sequences or elements of a second species, e.g., murine Fab domains.

In some embodiments the monoclonal antibodies are ‘humanized’. Humanization of antibodies comprises replacing antigen-non-specific sequences of one species, e.g., CDRs of human IgG, with antigen-specific sequences of another species, e.g., CDRs of murine monoclonal IgG, to lower the chance of an immune response once the therapeutic antibody is introduced into humans.

### Antigen

An antigen is defined as any molecule or compound that can bind in a specific manner to an antibody. Antigens include but are not limited to allergens, cancer antigens, microbial antigens, and autoimmune disease antigens. Antigens can be compounds or molecules that are present in the circulation of a subject. An antigen may be a heterologous target (for example a target on a bacterium, virus or other pathogen) or may be expressed on the surface of at least one cell or tissue. Embodiments of antigens are proteins, peptides, polypeptides, nucleic acids, polysaccharides, lipids and synthetic compounds.

In some embodiments the antigen is an antibody.

### Sample

A sample is any solution, fluid, liquid or solid that contains, or may contain, one or more antigens of interest. A reference sample contains one or more antigens of interest. A test sample may contain one or more antigens of interest. In some embodiments a sample is a biological sample. A biological sample can be a tissue or a biological fluid. Biological fluids include blood, serum, plasma, urine, saliva, milk, semen, tears, sweat, bile, cerebrospinal fluid and mucus, but are not so limited. A biological sample can be a tissue culture product, e.g., a tissue culture supernatant or a tissue culture lysate. In some embodiments the sample is lysed or otherwise prepared to allow for binding of the antigen to the antibody coupled to the antigen-capture substrate.

Contacting the sample is defined as bringing the sample in close enough proximity to allow for binding between an antigen of the sample and an antibody, specific for the antigen, which antibody is coupled to the antigen-capture substrate of the invention.

### Specific Binding of the Antigen

Specific binding of an antigen to an antibody coupled to the antigen-capture substrate is defined as binding with a dissociation constant (K_{D}) of 10⁻⁵ to 10⁻¹² M (moles/litre) or less. The binding is non-specific if the K_{D} is greater than 10⁻⁵ M. In some embodiments of specific binding the antigen remains bound to the antibody after the sample that contained the antigen has been removed and the antigen-capture substrate with the bound antigen has been washed.

### ELISA

Enzyme-linked immunosorbent assay (ELISA) is a well-known type of assay used for the detection of various antigens. The invention embraces all immunosorbent assays in which an antigen is specifically bound to an antibody coupled to an antigen-capture substrate of the invention. In some embodiments the antigen-capture substrate comprises an antibody.

A first step of an ELISA performed according to a method of the invention comprises providing an antigen-capture substrate of the invention, which substrate is coupled to or includes a primary antibody specific for an antigen of interest, and which for example is in the form of a multiwell plate, and blocking the substrates using a buffer containing a high concentration of irrelevant protein, such as bovine serum albumin or casein. The blocking step ensures that any uncoated areas of the surface will be occupied with non-reactive protein. Excess blocking agent is then removed by one or more washes. Once the surface is blocked, a sample containing the antigen of interest, or a sample to be tested for the antigen of interest, can be contacted with the antigen-capture substrate and is allowed to incubate under conditions and for an amount of time suitable to permit specific binding of the antigen by the primary antibody. Such conditions and amount of time can be, for example, room temperature for 3-4 hours, or 4° C. for 10-16 hours. Excess sample is then removed by one or more washes. As a next step, a secondary antibody, also specific for the antigen, is contacted with the antigen-capture substrate and is allowed to incubate under conditions and for an amount of time suitable to permit specific binding of the antigen by the secondary antibody. Such conditions and amount of time can be, for example, antibody at 1-10 microgram/ml, room temperature for 1-4 hours, and 4° C. for 10-16 hours. In some embodiments the secondary antibody is connected to a fluorescent tag or to an metabolizing enzyme, allowing for the detection of bound antigen. Alternatively, bound antigen can be determined by contacting the secondary antibody with a labeled tertiary antibody.

The above-described ELISA is referred to as a sandwich ELISA as the antigen is sandwiched between two antibodies (the antibody of the antigen-capture substrate and the secondary antigen). The invention is not limited to sandwich ELISAs and embraces any ELISA that comprises an antigen-capture substrate. In some embodiments the antigen is coupled to a detection label, obviating the need for a secondary antibody. In some embodiments the ELISA comprises a tertiary antibody, specific to the capture-antigen substrate, which allows for the determination of the amount of capture-antigen substrate that did not bind antigen.

In some embodiments determining if an antigen is bound an antibody-capture substrate comprises detecting the bound antigen. To allow for detection of the bound antigen, the secondary or tertiary antibody will have an associated label to allow for its detection. In some embodiments the label is an enzyme that will generate color development upon incubating with an appropriate chromogenic substrate, which allows for determining the presence and/or amount of antigen bound to the antibody through an absorbance measurement. Measurement of the amount of bound antigen through an absorbance measurement is routine in the art. Non-limiting examples of enzymes are urease, glucose oxidase, alkaline phosphatase, and hydrogen peroxidase. Non-limiting examples of chromogenic substrates include urea, bromocresol purple, and 2,2′-azino-di-(3-ethyl-benzthiazoline-6-sulfonic acid) (ABTS) and H₂O₂, in the case of peroxidase as the enzyme label. Quantification is then achieved by measuring the degree of color generation, e.g., using a spectrophotometer.

Antibodies also may be coupled to specific labeling agents or imaging agents, including, but not limited to a molecule preferably selected from the group consisting of fluorescent, enzyme, radioactive, metallic, biotin, chemiluminescent, bioluminescent, chromophore, or colored, etc. In some aspects of the invention, a label may be a combination of the foregoing molecule types.

In some embodiments antigen bound to a capture antibody is detected through a fluorescence measurement. Fluorescence measurements are based on the excitation of a fluorescent label or fluorescent agent by a light source which results in the emission of light with a lower energy level which is detected. Measurement of the amount of bound antigen through a fluorescence measurement is routine in the art.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Antibody Orientation Assay

A primary antibody (human IgG) was applied to an antigen-capture substrate in the form of a 96-well microtiter plate. The primary antibody was incubated and allowed to bind to the antigen-capture substrate, after which any unbound antibody was removed by washing with a detergent buffer.

The plate was then blocked using a buffer containing a high concentration of protein, such as bovine serum albumin or casein. The blocking step ensures that any part of the polymer surface that does not contain antigen-capture substrate will be blocked with neutral, non-reactive protein so as not to interfere with subsequent steps within the assay. The blocking buffer was applied at three times the volume of the primary antibody; ensuring that the part of the well that was not exposed to the primary antibody was completed coated with non-reactive protein. After the blocking step was completed, a second wash step was performed.

Two secondary antibodies were subsequently applied at the same volume as the primary antibody, one specific for the Fab domain of human IgG, the other specific for the Fc region of human IgG. Both antibodies were labeled with fluorophores such as FITC (fluorescein isothiocyanate) or TRITC (tetramethylrhodamine isothiocyanate). The secondary antibodies were incubated on the plate and at the end of the incubation period the plates were washed as previously described and fluorescence intensity was determined for each well of the 96-well plate. By comparing the fluorescence intensity of the anti-Fab- and anti-Fc-labeled antibodies to each other, and to appropriate controls, the proportion of antibodies in the correct orientation was determined.

If the Fab- and Fc-specific secondary antibodies are both labeled with FITC, then the assay can be divided with one half of the plate receiving the Fab-specific antibodies and the other half the Fc-specific antibodies. If the Fab- and Fc-specific secondary antibodies are labeled with different fluorophores, then they may then be combined in the same well.

To determine what, if any, effect Protein G′ in combination with different polymers had on antibody orientation, a series of polymers was evaluated (Table 1). The polymers were applied to 96-well microtiter plates using solution casting.

TABLE 1
Selected Polymers
Polymer

Poly(acrylic acid)
Poly(styrenesulfonate)/poly(2,3-dihydrothieno(3,4-b)-dioxin)
Poly(aniline)
Poly(methyl methacrylate)
Poly(styrene-co-methyl methacrylate)
Poly(styrene)
Poly(acrylic acid)

### Example 1

### Solution Casting

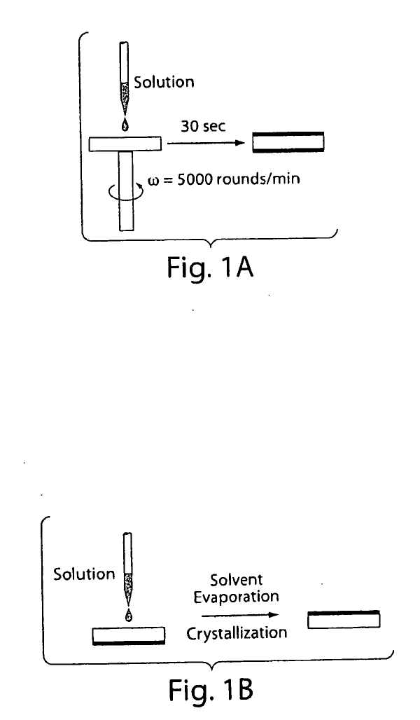

Solution casting is a method in which thin polymer films are developed on a solid substrate (41). Polymers were solubilized in an appropriate solvent then applied to the substrate. The polymer was subsequently dispersed in a suitable manner (e.g., by mechanical or manual spreading) resulting in a layer of desired thickness. As a last step the solvent was evaporated leaving a thin polymer film (FIG. 1).

### Example 2

### Polymer Preparation

Polymers were solubilized in the appropriate solvent at a concentration of 0.5-1 mg/ml (Table 2) by slow addition of the dry polymer to the solvent with rapid stirring.

TABLE 2
Polymer Concentrations and Solvents

| Polymer | Concentration | Solvent |
|---|---|---|
| Poly(acrylic acid) | 1 mg/ml | Deionized |
| | | Water |
| Poly(styrenesulfonate)/poly(2,3- | 1 mg/ml | Deionized |
| dihydrothieno(3,4-b)-dioxin) | | Water |
| Poly(aniline) | 1 mg/ml | Deionized |
| | | Water |
| Poly(methyl methacrylate) | 0.5 mg/ml | Acetone |
| Poly(styrene-co-methyl methacrylate) | 0.5 mg/ml | Acetone |
| Poly(styrene) | 0.5 mg/ml | Toluene |

### Example 3

### Generation of Thin Films

Solubilized polymers were applied to the wells of a 96-well assay plate composed of polypropylene (Fisher Scientific; Waltham, Mass.), selected due to the imperviousness of polypropylene to the various solvents used in these experiments, at a volume of 50 microliters per well. The plates were placed in a chemical fume hood and rotated for 12 hours to permit solvent evaporation and film formation. Water-soluble polymers were also subjected to gentle drying at 50° C. for two hours. The plates were subsequently washed with one volume of a 1% sodium dodecyl sulfate (SDS) solution in phosphate buffered saline (PBS), pH 7.4, rinsed with three volumes of PBS and allowed to dry at room temperature for 24 hours, resulting in the formation of thin well-shaped polymer films

Four different commercially available 96-well polystyrene plates were used as controls: Polystyrene (Fisher Scientific), Ultra-high binding polystyrene (Immulon; Dynex Technologies, Inc., Chantilly, Va.), High-binding polystyrene (Immulon), and Medium-binding polystyrene (Immulon).

### Example 4

### Protein G′ Coating

Protein G′ (Sigma; St. Louis, Mo.) was diluted to a final concentration of 1 microgram/ml in PBS and applied to the experimental and control plates at a volume of 50 microliters/well. The plates were allowed to incubate for 12-24 hours at 4° C., then washed with 1 volume of 1% SDS in PBS and rinsed with 3 volumes of PBS and blotted with lint-free tissue.

### Example 5

### Primary Antibody Addition

Human IgG (Sigma) was diluted to a final concentration of 5 microgram/ml in PBS (pH 7.4), and applied to all wells of the experimental and control plates at a volume of 50 microliters/well. The plates were incubated for 12 hours at 4° C., then washed with 1 volume of 1% SDS in PBS and rinsed with 3 volumes of PBS then blotted with lint-free tissue.

A blocking buffer consisting of 5% (w/v) non-fat dried milk (NFDM) in PBS was prepared and added to all wells of all plates at a volume of 150 microliters per well. The plates were incubated with the blocking buffer at room temperature for 2 hours, then washed with 1 volume of 1% SDS in PBS and rinsed with 3 volumes of PBS.

### Example 6

### Secondary Antibody Addition

FITC-conjugated Fab-specific antibody (Sigma) and FITC-conjugated Fc-specific antibody (Sigma) were diluted to concentrations of 10 microgram/ml each in PBS at a volume of 100 microliters per well. A 1:1 serial dilution scheme in PBS was carried out for each antibody conjugate in the wells of subsequent rows (which contained 50 microliters of PBS each), resulting in concentrations of 10, 5, 2.5 and 1.25 microgram/ml. The plates were incubated for 1 hour at ambient temperature, washed with 1 volume of 1% SDS in PBS, and rinsed with 3 volumes of PBS and read for fluorescence on a BioTek Plate Reader (BioTek Corporation, Winooski, V.T.).

FITC-conjugated Fab-specific antibody (Sigma) and TRITC-conjugated Fc-specific antibody (Sigma) were diluted together to concentrations of 10 microgram/ml each in PBS at a volume of 100 microliters per well. A 1:1 serial dilution scheme in PBS was carried out for each antibody conjugate in the wells of subsequent rows (which contained 50 microliters of PBS each), resulting in concentrations of 10, 5, 2.5 and 1.25 microgram/ml. The plates were incubated for 1 hour at ambient temperature, washed with 1 volume of 1 SDS in 1×PBS, and rinsed with 3 volumes of PBS. Fluorescence intensity was quantitated on a BioTek Synergy™ HT Multi-Detection Microplate Reader. This instrument is capable of detection via absorbance, fluorescence and luminescence. For those plates containing FITC-labeled Fab- and Fc-specific antibodies the plate is read once with the parameters described in. For those plates containing FITC-labeled Fab-/TRITC-Labeled Fc-specific antibodies the plates were first read with the FITC parameters and then a second time with the parameters for TRITC.

An Olympus FV1000 Confocal Fluorescence Microscope (Nashua, N.H.) was used to determine fluorescent intensity on a Protein G′ coated polymer thin-film with human IgG complexed with FITC-labeled Fab- and Fc-specific antibodies and compared against a control plate. Fluorescence intensity data points corresponding to different areas of the wells were collected and graphed and images, each corresponding to a data point are also collected and converted to black- and white images so that the fluorophores images may be quantified and mapped.

### Example 7

### Initial Polymer Screening

Four polymers: Poly(methyl methacrylate) (PMMA), Poly(styrenesulfonate)/poly(2,3-dihydrothieno(3,4-b)-dioxin) (PPD), Poly(acrylic acid) (PAA) and Polyaniline (PANI) were adsorbed onto 96-well polypropylene plates (Fisher Scientific) by solution casting as described above.

One half of each plate (corresponding to 48 wells) was coated with Protein G′ at a concentration of 1 microgram/ml at 50 microliters per well and allowed to incubate for 12 hours at 4° C., along with a control plate consisting of high-binding polystyrene (Fisher Scientific). The plates were washed, coated with Human IgG (Sigma) at a concentration of 5 microgram/ml and incubated for 12 hours at 4° C. The plates were washed again, and blocked with a 1% solution of non-fat dried milk in phosphate buffered saline (pH 7.4). After another wash, FITC-conjugated Fab-specific antibody (Sigma) and FITC-conjugated Fc-specific antibody (Sigma) were diluted to concentrations of 10 microgram/ml each in PBS at a volume of 100 microliters per well.

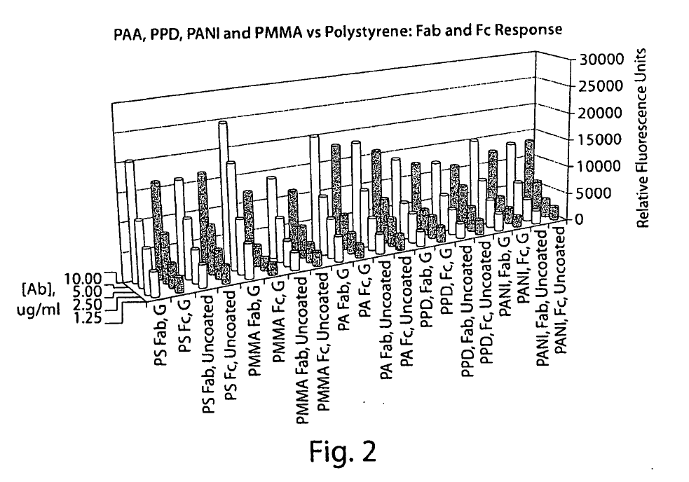

A 1:1 serial dilution scheme in PBS was carried out for each antibody conjugate resulting in concentrations of 10, 5, 2.5 and 1.25 microgram/ml. The plates were incubated for 1 hour at room temperature and read for fluorescence on a BioTek Synergy™ HT Multi-Detection Microplate Reader. The data were collected and saved to an Excel spreadsheet. The data was analyzed for mean (n=18 per dilution point), and standard deviation using Excel statistical functions. The percent coefficient of variation (% CV) was calculated as % CV=(Mean/Standard Deviation)×100%. The data are provided in Table 3 and are shown in FIG. 2.

TABLE 3
Screening Results

| | MEAN | | | | STDEV | | | | % CV | | | |
|---|---|---|---|---|---|---|---|---|---|---|---|---|
| [C] | Fab G | Fc G | Fab NO G | Fc NO G | Fab G | Fc G | Fab NO G | Fc NO G | Fab G | Fc G | Fab NO G | Fc NO G |
| PS | | | | | | | | | | | | |
| 10 | 20590 | 16606 | 16506 | 16878 | 1010 | 1453 | 1831 | 1259 | 5 | 9 | 11 | 7 |
| 5 | 11426 | 8523 | 10423 | 8566 | 1295 | 436 | 499 | 818 | 11 | 5 | 5 | 10 |
| 2.5 | 7506 | 4109 | 5857 | 5008 | 427 | 268 | 1277 | 856 | 6 | 7 | 22 | 17 |
| 1.25 | 4426 | 2569 | 4001 | 3091 | 387 | 282 | 824 | 611 | 9 | 11 | 21 | 20 |
| PAA | | | | | | | | | | | | |
| 10 | 20264 | 18247 | 18194 | 16125 | 2794 | 920 | 896 | 575 | 14 | 5 | 5 | 4 |
| 5 | 10409 | 6427 | 10181 | 7995 | 3172 | 150 | 148 | 239 | 30 | 2 | 1 | 3 |
| 2.5 | 6938 | 3615 | 5846 | 5046 | 647 | 266 | 441 | 504 | 9 | 7 | 8 | 10 |
| 1.25 | 4645 | 2367 | 3665 | 2710 | 494 | 286 | 183 | 276 | 11 | 12 | 5 | 10 |
| PPD | | | | | | | | | | | | |
| 10 | 14038 | 12578 | 12273 | 11024 | 227 | 276 | 1936 | 443 | 2 | 2 | 16 | 4 |
| 5 | 6844 | 4805 | 6865 | 7814 | 384 | 151 | 813 | 868 | 6 | 3 | 12 | 11 |
| 2.5 | 5419 | 4144 | 4821 | 4577 | 972 | 159 | 1196 | 216 | 18 | 4 | 25 | 5 |
| 1.25 | 2764 | 2817 | 2798 | 2725 | 560 | 199 | 791 | 244 | 20 | 7 | 28 | 9 |
| PANI | | | | | | | | | | | | |
| 10 | 15392 | 12518 | 13702 | 13495 | 3408 | 498 | 1792 | 750 | 22 | 4 | 13 | 6 |
| 5 | 8398 | 4568 | 6741 | 6204 | 278 | 1063 | 1930 | 551 | 3 | 23 | 29 | 9 |
| 2.5 | 5356 | 3108 | 4181 | 3451 | 96 | 424 | 920 | 1041 | 2 | 14 | 22 | 30 |
| 1.25 | 3418 | 2016 | 2740 | 2569 | 334 | 538 | 686 | 113 | 10 | 27 | 25 | 4 |
| PMMA | | | | | | | | | | | | |
| 10 | 24907 | 12379 | 14318 | 11414 | 777 | 496 | 1438 | 407 | 3 | 4 | 10 | 4 |
| 5 | 18521 | 3769 | 7871 | 5736 | 301 | 555 | 408 | 209 | 2 | 15 | 5 | 4 |
| 2.5 | 9675 | 2181 | 4491 | 3353 | 235 | 163 | 269 | 335 | 2 | 7 | 6 | 10 |
| 1.25 | 6226 | 1844 | 3179 | 2476 | 282 | 199 | 125 | 144 | 5 | 11 | 4 | 6 |

Of the five polymers, PMMA in combination with Protein G′ showed the most favorable response when compared with the polystyrene control plates; that is, the highest Fab response and the lowest Fc response.

### Example 8

### Comparison PMMA with PS/PMMA Co-Polymer and PS Control

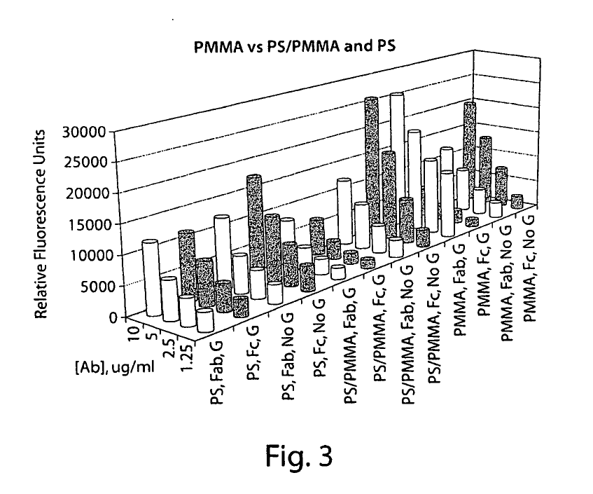

PMMA was compared to a PS/PMMA copolymer and a PS control plate by adsorbing the PMMA and PS/PMMA co-polymer to the wells of a 96-well polypropylene plate (Fisher Scientific) by solution casting and carrying out the antibody orientation assay as previously described. The data were collected and analyzed as described in Example 7 (n=18). The data are provided in Table 4 and FIG. 3.

TABLE 4
PMMA vs PS/PMMA and Polystyrene

| | MEAN | | | | STDEV | | | | % CV | | | |
|---|---|---|---|---|---|---|---|---|---|---|---|---|
| | Fab G | Fc G | Fab NO G | Fc NO G | Fab G | Fc G | Fab NO G | Fc NO G | Fab G | Fc G | Fab NO G | Fc NO G |
| PS | | | | | | | | | | | | |
| 10 | 12067 | 11683 | 12322 | 17543 | 989 | 499 | 452 | 702 | 8 | 4 | 4 | 4 |
| 5 | 6798 | 7724 | 6690 | 11521 | 222 | 272 | 619 | 139 | 3 | 4 | 9 | 1 |
| 2.5 | 4471 | 4775 | 4917 | 7321 | 172 | 521 | 389 | 315 | 4 | 11 | 8 | 4 |
| 1.25 | 3213 | 2985 | 3271 | 4437 | 256 | 752 | 185 | 712 | 8 | 25 | 6 | 16 |
| PMMA | | | | | | | | | | | | |
| 10 | 25629 | 5687 | 12172 | 20087 | 2628 | 277 | 1749 | 1064 | 10 | 5 | 14 | 5 |
| 5 | 18976 | 2723 | 8264 | 13378 | 864 | 450 | 693 | 649 | 5 | 17 | 8 | 5 |
| 2.5 | 13845 | 2466 | 4951 | 7553 | 619 | 284 | 340 | 670 | 4 | 12 | 7 | 9 |
| 1.25 | 12201 | 1370 | 3078 | 2377 | 656 | 306 | 755 | 428 | 5 | 22 | 25 | 18 |
| PS/PMMA | | | | | | | | | | | | |
| 10 | 8036 | 6384 | 12172 | 26011 | 1111 | 947 | 1749 | 622 | 14 | 15 | 14 | 2 |
| 5 | 3914 | 3285 | 8264 | 16360 | 1542 | 275 | 693 | 2670 | 39 | 8 | 8 | 16 |
| 2.5 | 2709 | 1788 | 4951 | 8096 | 532 | 387 | 340 | 1772 | 20 | 22 | 7 | 22 |
| 1.25 | 1750 | 1600 | 3078 | 3057 | 200 | 135 | 755 | 453 | 11 | 8 | 25 | 15 |

### Example 9

### PMMA Compared with UHB, HB, MB

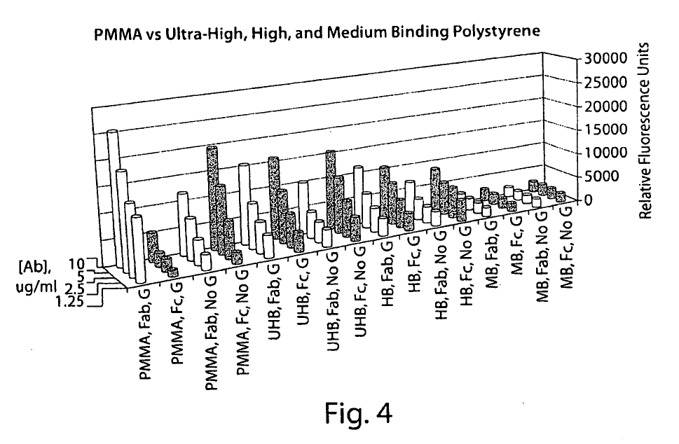

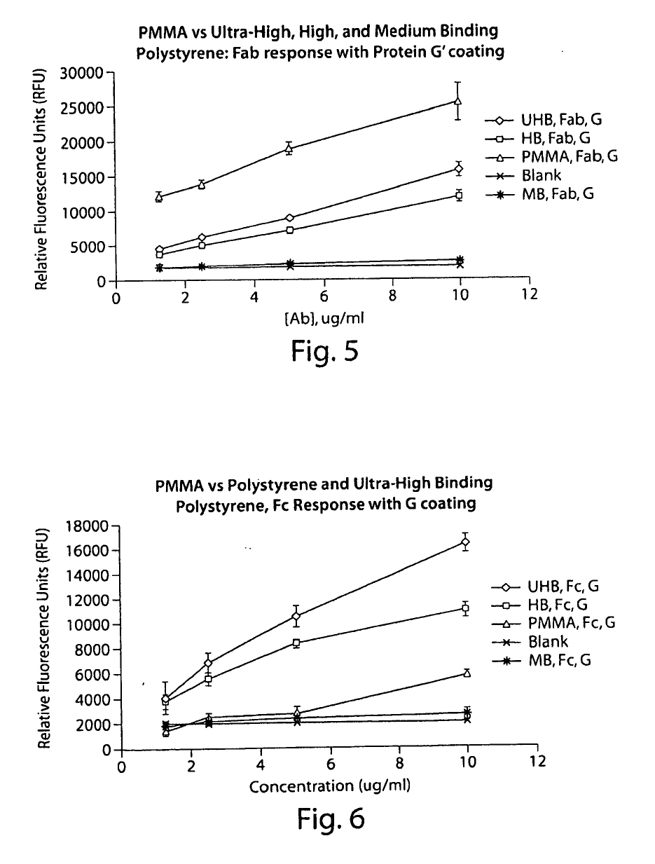

PMMA was compared against ultra-high binding (UHB), high-binding (HB), and medium-binding (MB) control plates (Immulon) by adsorbing the PMMA to the wells of a 96-well polypropylene plate (Fisher Scientific) by solution casting and carrying out the antibody orientation assay as described in Example 7. The data were collected and analyzed as described in Example 7 (n=18). The data are provided in Table 5 and FIGS. 4-6.

TABLE 5
PMMA vs UHB, HB, and MB Polystyrene

| | MEAN | | | | STDEV | | | | % CV | | | |
|---|---|---|---|---|---|---|---|---|---|---|---|---|
| | Fab G | Fc G | Fab NO G | Fc NO G | Fab G | Fc G | Fab NO G | Fc NO G | Fab G | Fc G | Fab NO G | Fc NO G |
| PMMA | | | | | | | | | | | | |
| 10 | 25629 | 5687 | 12172 | 20087 | 2628 | 277 | 1749 | 1064 | 10 | 5 | 14 | 5 |
| 5 | 18976 | 2723 | 8264 | 13378 | 864 | 450 | 693 | 649 | 5 | 17 | 8 | 5 |
| 2.5 | 13845 | 2466 | 4951 | 7553 | 619 | 284 | 340 | 670 | 4 | 12 | 7 | 9 |
| 1.25 | 12201 | 1370 | 3078 | 2377 | 656 | 306 | 755 | 428 | 5 | 22 | 25 | 18 |
| UHB | | | | | | | | | | | | |
| 10 | 15919 | 16350 | 10577 | 15943 | 1048 | 709 | 1315 | 6845 | 7 | 4 | 12 | 43 |
| 5 | 9012 | 10470 | 5375 | 11256 | 307 | 852 | 1552 | 386 | 3 | 8 | 29 | 3 |
| 2.5 | 6230 | 6829 | 4272 | 7336 | 244 | 799 | 931 | 539 | 4 | 12 | 22 | 7 |
| 1.25 | 4539 | 4048 | 3622 | 5029 | 252 | 1311 | 212 | 499 | 6 | 32 | 6 | 10 |
| HB | | | | | | | | | | | | |
| 10 | 11913 | 10919 | 6993 | 8937 | 758 | 584 | 881 | 1707 | 6 | 5 | 13 | 19 |
| 5 | 7133 | 8253 | 3982 | 6602 | 458 | 337 | 401 | 296 | 6 | 4 | 10 | 4 |
| 2.5 | 4977 | 5484 | 3244 | 6234 | 341 | 496 | 194 | 1144 | 7 | 9 | 6 | 18 |
| 1.25 | 3722 | 3699 | 3201 | 4399 | 304 | 585 | 473 | 683 | 8 | 16 | 15 | 16 |
| MB | | | | | | | | | | | | |
| 10 | 2526 | 2524 | 2154 | 2737 | 211 | 474 | 332 | 824 | 8 | 19 | 15 | 30 |
| 5 | 2267 | 2341 | 1867 | 2859 | 167 | 153 | 55 | 174 | 7 | 7 | 3 | 6 |
| 2.5 | 1994 | 2128 | 1775 | 2735 | 106 | 31 | 50 | 854 | 5 | 1 | 3 | 31 |
| 1.25 | 1935 | 1843 | 1779 | 2219 | 420 | 85 | 256 | 393 | 22 | 5 | 14 | 18 |

### Example 10

### PMMA Compared with PS Film

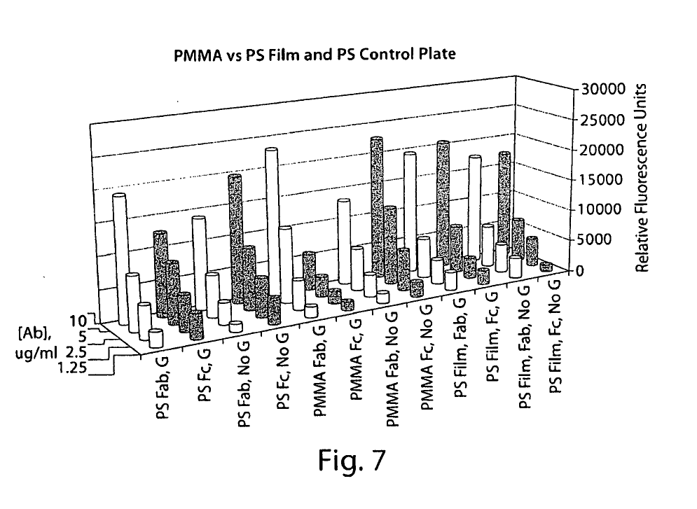

PMMA was compared against a PS film and a PS control plate by adsorbing the PMMA and PS to the wells of a 96-well polypropylene plate (Fisher Scientific) by solution casting and carrying out the antibody orientation assay as previously described. The data were collected and analyzed as described above. The data are provided in Table 6 and FIG. 7.

TABLE 6
PMMA vs PS Film and PS Control

| | MEAN | | | | STDEV | | | | % CV | | | |
|---|---|---|---|---|---|---|---|---|---|---|---|---|
| | Fab G | Fc G | Fab NO G | Fc NO G | Fab G | Fc G | Fab NO G | Fc NO G | Fab G | Fc G | Fab NO G | Fc NO G |
| PS | | | | | | | | | | | | |
| 10 | 19547 | 12973 | 14352 | 20073 | 1025 | 565 | 589 | 2159 | 5 | 4 | 4 | 11 |
| 5 | 8580 | 9414 | 6593 | 9698 | 892 | 720 | 286 | 573 | 10 | 8 | 4 | 6 |
| 2.5 | 5222 | 5717 | 3486 | 6067 | 607 | 508 | 256 | 545 | 12 | 9 | 7 | 9 |
| 1.25 | 2397 | 4037 | 1326 | 4109 | 250 | 216 | 279 | 216 | 10 | 5 | 21 | 5 |
| PS Film | | | | | | | | | | | | |
| 10 | 19800 | 20838 | 17658 | 17526 | 3516 | 3499 | 3170 | 1111 | 18 | 17 | 18 | 6 |
| 5 | 6418 | 7559 | 6679 | 6650 | 2308 | 6591 | 485 | 668 | 36 | 87 | 7 | 10 |
| 2.5 | 3947 | 3299 | 4561 | 4443 | 1139 | 827 | 310 | 690 | 29 | 25 | 7 | 16 |
| 1.25 | 2907 | 2188 | 3380 | 1212 | 678 | 937 | 316 | 116 | 23 | 43 | 9 | 10 |
| PMMA | | | | | | | | | | | | |
| 10 | 23279 | 5856 | 13617 | 22969 | 901 | 478 | 1033 | 1298 | 4 | 8 | 8 | 6 |
| 5 | 11738 | 3200 | 6633 | 12459 | 913 | 323 | 446 | 396 | 8 | 10 | 7 | 3 |
| 2.5 | 4721 | 1901 | 3478 | 6421 | 855 | 457 | 247 | 323 | 18 | 24 | 7 | 5 |
| 1.25 | 1735 | 1397 | 1655 | 2385 | 318 | 287 | 222 | 624 | 18 | 21 | 13 | 26 |

### Example 11

### PMMA-FITC/TRITC Assay

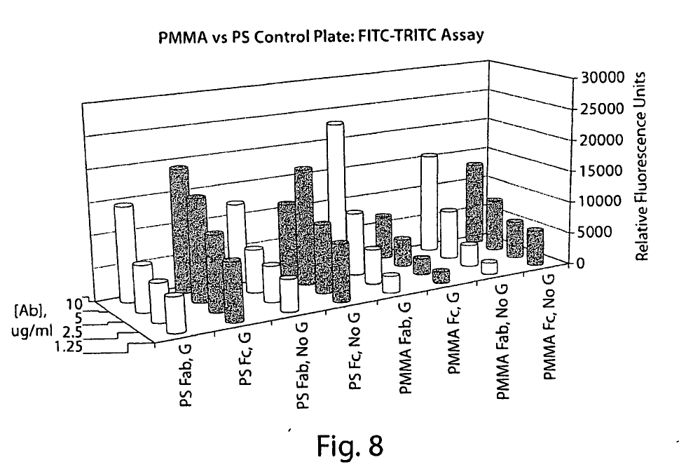

PMMA was compared against a PS control plate by adsorbing the PMMA to the wells of a 96-well polypropylene plate (Fisher Scientific) by solution casting and carrying out the antibody orientation assay as described above, with the following changes: FITC-conjugated Fab-specific antibody (Sigma) and TRITC-conjugated FC-specific antibody (Sigma) were diluted together to concentrations of 10 microgram/ml each in PBS at a volume of 100 microliters per well. A 1:1 serial dilution scheme in PBS was carried out for each antibody conjugate in the wells of subsequent rows, resulting in concentrations of 10, 5, 2.5 and 1.25 microgram/ml. The data were collected and analyzed as described above. The data are provided in Table 7 and FIG. 8 (n=18).

TABLE 7
PMMA vs PS Control, FITC-TRITC Assay

| | MEAN | | | | STDEV | | | | % CV | | | |
|---|---|---|---|---|---|---|---|---|---|---|---|---|
| | Fab G | Fab NO G | Fc G | Fc NO G | Fab G | Fab NO G | Fc G | Fc NO G | Fab G | Fab NO G | Fc G | Fc NO G |
| PS | | | | | | | | | | | | |
| 10 | 14952 | 12773 | 19384 | 11459 | 1091 | 405 | 1669 | 2157 | 14 | 6 | 9 | 10 |
| 5 | 7322 | 6884 | 16152 | 18140 | 327 | 677 | 1406 | 953 | 4 | 10 | 9 | 5 |
| 2.5 | 6075 | 5722 | 11787 | 10708 | 458 | 509 | 752 | 1428 | 8 | 9 | 6 | 13 |
| 1.25 | 5525 | 5106 | 9117 | 9006 | 528 | 391 | 1280 | 716 | 10 | 8 | 14 | 8 |
| PMMA | | | | | | | | | | | | |
| 10 | 23272 | 15979 | 6934 | 13407 | 1966 | 2324 | 1207 | 1853 | 8 | 15 | 17 | 14 |
| 5 | 9883 | 7842 | 4466 | 8250 | 881 | 903 | 2015 | 2082 | 9 | 12 | 31 | 25 |
| 2.5 | 5352 | 3530 | 2609 | 6001 | 287 | 417 | 1338 | 1603 | 5 | 12 | 29 | 27 |
| 1.25 | 2612 | 1818 | 1854 | 5816 | 260 | 385 | 1203 | 1068 | 10 | 21 | 26 | 18 |

### Example 12

### PMMA vs. PS Control: Confocal Fluorescence Microscopy

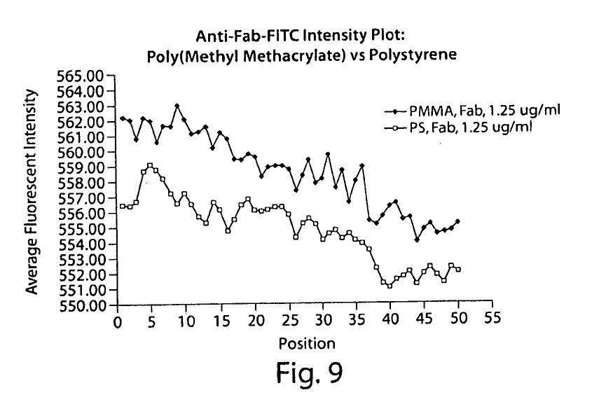

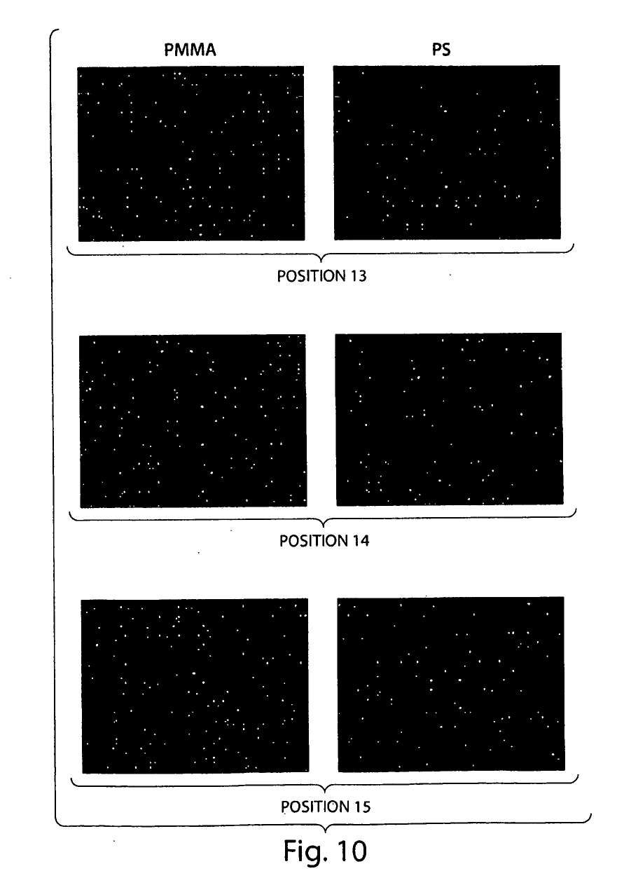

PMMA was compared against an ultra-high binding PS control plate (Immulon) by adsorbing the PMMA to the wells of a 96-well polypropylene plate (Fisher Scientific) by solution casting and carrying out the antibody orientation assay as previously described with the following changes: The identity and concentration was limited to 1.25 microgram/ml of FITC-labeled Fab-specific antibodies for both the PMMA and PS plates. An Olympus FV1000 Confocal Fluorescence Microscope (Olympus) was used to determine fluorescent intensity on both plates (n=2), over 50 positions (Table 8). These data are presented in FIG. 9 and photographs of each position were taken and converted to black-and-white TIFF format to visualize the fluorophores (FIG. 10).

TABLE 8
Anti-Fab-FITC Intensity Plot: Poly(Methyl Methacrylate) vs
Polystyrene

| Position | PMMA | PS |
|---|---|---|
| 1 | 562.24 | 556.44 |
| 2 | 562.07 | 556.39 |
| 3 | 560.84 | 556.66 |
| 4 | 562.21 | 558.66 |
| 5 | 562.00 | 559.12 |
| 6 | 560.62 | 558.74 |
| 7 | 561.69 | 558.19 |
| 8 | 561.64 | 557.19 |
| 9 | 563.04 | 556.55 |
| 10 | 562.14 | 557.21 |
| 11 | 561.19 | 556.49 |
| 12 | 561.29 | 555.68 |
| 13 | 561.62 | 555.28 |
| 14 | 560.25 | 556.65 |
| 15 | 561.24 | 556.13 |
| 16 | 560.83 | 554.72 |
| 17 | 559.48 | 555.49 |
| 18 | 559.43 | 556.42 |
| 19 | 559.81 | 556.84 |
| 20 | 559.65 | 556.07 |
| 21 | 558.37 | 556.02 |
| 22 | 558.99 | 556.17 |
| 23 | 559.06 | 556.30 |
| 24 | 559.05 | 556.32 |
| 25 | 558.81 | 555.77 |
| 26 | 557.44 | 554.30 |
| 27 | 558.39 | 555.22 |
| 28 | 559.43 | 555.54 |
| 29 | 557.91 | 555.17 |
| 30 | 558.18 | 554.13 |
| 31 | 559.78 | 554.57 |
| 32 | 557.62 | 554.74 |
| 33 | 558.74 | 554.25 |
| 34 | 556.63 | 554.56 |
| 35 | 558.03 | 554.09 |
| 36 | 558.95 | 553.92 |
| 37 | 555.46 | 553.47 |
| 38 | 555.19 | 552.26 |
| 39 | 555.71 | 551.25 |
| 40 | 556.38 | 551.03 |
| 41 | 556.59 | 551.60 |
| 42 | 555.52 | 551.74 |
| 43 | 555.64 | 552.03 |
| 44 | 554.08 | 551.23 |
| 45 | 554.87 | 551.94 |
| 46 | 555.19 | 552.34 |
| 47 | 554.59 | 551.77 |
| 48 | 554.70 | 551.33 |
| 49 | 554.80 | 552.31 |
| 50 | 555.23 | 552.03 |

### REFERENCES

1. Lindl, T. 1996. Development of human monoclonal antibodies: A review. Cytotechnology. Vol. 21, No. 3, 183-193.
2. Papac, D. I., Hoyes J and Tomer, K. B. 1994. Epitope mapping of the gastrin-releasing peptide/anti-bombesin monoclonal antibody complex by proteolysis followed by matrix-assisted laser desorption inonization mass spectrometry. Protein Science, Vol. 3, 1485-1492.
3. Breeveld, F. C. 2000. Therapeutic monoclonal antibodies. The Lancet. Vol. 355, No. 9205, 735-740.
4. Guyre, P. M., Graziano, R. F, Vance, B. A, Morganelli P. M and Fanger, M. W. 1989. Monoclonal antibodies that bind to distinct epitopes on Fc gamma RI are able to trigger receptor function. J. Immunol. Vol 143, No. 5, 1650-1655.
5. Yang, X. D., Jia, X., Corvalan, J. R. F., Wang, P., Davis, C. G and Jakobovits, A. 1999. Eradication of established tumors by a fully human monoclonal antibody to the epidermal growth factor receptor without concomitant hemotherapy. Cancer Research. Vol. 59, 1236-1243.
6. Milstein, C. 1986. From antibody structure to immunological diversification of immune response. Science. Vol. 231. no. 4743, 1261-1268.
7. Kilar, F. and Zavodsky, P. 1987. Non-covalent interactions between Fab and Fc regions in immunoglobulin G molecules. Eur. J. Biochem. Vol. 162, No. 1, 57-61.
8. Taschner, N., Mueller, S., Alumella, V. R., Goldie, K. N., Drake, A. F. Aebi, U. and Arvinte, T. 2001. Modulation of Antigenicity Related to Changes in Antibody Flexibility upon Lyophilization. J. Mol. Biol. Vol. 310, 169-179.
9. Roux, K. H., Strelets, L. and Michaelsen, T. E. 1997. Flexibility of human IgG subclasses. J. Immunol., Vol 159, No. 7, 3372-3382.
10. Goldstein, G. 1987. Overview of the development of Orthoclone OKT3: monoclonal antibody for therapeutic use in transplantation. Transplant. Proc. Vol. 19 No. 2 Suppl. 1, 1-6.
11. Lee L J, Yang S T, Lai S, Bai Y, Huang W C, Juang Y J. 2006. Microfluidic enzyme-linked immunosorbent assay technology. Adv. Clin Chem. Vol. 42, 255-295.
12. Howanitz P J. 1988. Immunoassay. Development and directions in antibody technology. Arch Pathol Lab Med. Vol. 112 No. 8 771-774.
13. Crowther, J R. 1995. ELISA Theory and Practice. Humana Press, New Jersey. Pp. 36-43.
14. Oelschlaeger P, Srikant-Iyer S, Lange S, Schmitt J, Schmid R D. 2002. Fluorophor-linked immunosorbent assay: a time- and cost-saving method for the characterization of antibody fragments using a fusion protein of a single-chain antibody fragment and enhanced green fluorescent protein. Anal Biochem. Vol. 309 No. 1, 27-34
15. Gibbs, J. 2001. Elisa Technical Bulletin No. 5. Corning Life Sciences, Kennebunkport Me. pp. 7-10.
16. Crowther, J R. 1995. ELISA Theory and Practice. Humana Press, New Jersey. pp 63-64.
17. Pearson J E; Gill A; Vadgama P. 2000. Analytical aspects of biosensors. Annala Clin. Biochem. Vol. 37 No. 2, 119-145.
18. Fjield T, Sorrentino V, Cline H, Hynynen K. 1997. Design and experimental verification of thin acoustic lenses for the coagulation of large tissue volumes. Phys. Med. Biol. Vol. 42, No. 12, 2341-54.
19. Gibbs, J. 2001. Elisa Technical Bulletin No. 5. Corning Life Sciences, Kennebunkport Me. p. 2.
20. Gibbs, J. 2001. Elisa Technical Bulletin No. 5. Corning Life Sciences, Kennebunkport Me. pp. 3-5.
21. Ahluwalia A, De Rossi D, Giusto G, Chen O, Papper V, Likhtenshtein G I. 2002. A fluorescent-photochrome method for the quantitative characterization of solid phase antibody orientation. Anal. Biochem. Vol. 305 No. 2, 121-34.
22. Oh S W, Moon J D, Lim H J, Park S Y, Kim T, Park J, Han M H, Snyder M, Choi E Y. 2005. Calixarene derivative as a tool for highly sensitive detection and oriented immobilization of proteins in a microarray format through noncovalent molecular interaction. FASEB J. Vo. 19 No. 10, 1335-7.
23. Taussig, M J and Landegren, U. 2003. Progress in antibody arrays. Targets, 2:4:169-176.
24. Doumit M E, Lonergan S M, Arbona J R, Killefer J, Koohmaraie M. 1996. Development of an enzyme-linked immunosorbent assay (ELISA) for quantification of skeletal muscle calpastatin. J Anim Sci. Vol. 74 No. 11, 2679-86.
25. Lu B, Smyth M R, O′Kennedy R. 1996. Oriented immobilization of antibodies and its applications in immunoassays and immunosensors. Analyst. Vol. 121 No. 3, 29-32.
26. Jung S H, Son H Y, Yuk J S, Jung J W, Kim K H, Lee C H, Hwang H, Ha K S. 2006. Oriented immobilization of antibodies by a self-assembled monolayer of 2-(biotinamido)ethanethiol for immunoarray preparation. Colloids Surf B Biointerfaces. Vol. 47 No. 1, 107-11.
27. Chen, S., Liu, L., Zhou, J., Jiangm S. 2006. Controlling antibody orientation on charged self-assembled monolayers. Langmuirm Vol. 19 No. 7, 2859-2864.
28. Bhatia S K, Shriver-Lake L C, Prior K J, Georger J H, Calvert J M, Bredehorst R, Ligler F S. 1989. Use of thiol-terminal silanes and heterobifunctional crosslinkers for immobilization of antibodies on silica surfaces. Anal Biochem. Vol. 178 No. 2, 408-13.
29. Subramanian A, Velander W H. 1996. Effect of antibody orientation on immunosorbent performance. J Mol. Recognit. Vol. 9, No. 5-6, 528-35.
30. Butler J E, Ni L, Nessler R, Joshi K S, Suter M, Rosenberg B. 1992. The physical and functional behavior of capture antibodies adsorbed on polystyrene. J Immunol Methods Vol. 24, No. 150, 77-90.
31. Akerstrom B, Brodin T, Reis K, Bjorck L. 1985. Protein G: a powerful tool for binding and detection of monoclonal and polyclonal antibodies. J Immunol. Vol. 135 No. 4, 2589-92.
32. Walker K N, Bottomley S P, Popplewell A G, Sutton B J, Gore M G. 1995. Equilibrium and pre-equilibrium fluorescence spectroscopic studies of the binding of a single-immunoglobulin-binding domain derived from protein G to the Fc fragment from human IgG1. Biochem J. Vol. 15 No. 310. 177-84.
33. Goward C R, Irons L I, Murphy J P, Atkinson T. 1991. The secondary structure of protein G′, a robust molecule. Biochem J. Vol. 1 No. 274, 503-7.
34. Vijayendran R A, Leckband D E. 2001. A quantitative assessment of heterogeneity for surface-immobilized proteins. Anal Chem. Vol. 73 No. 3 471-480.
35. Allcock, H R., Lampe, F W, Mark, J E. 2003. Contemporary Polymer Chemistry. Pearson Education Inc. Upper Saddle River, N.J. p. 526.
36. Allcock, H R., Lampe, F W, Mark, J E. 2003. Contemporary Polymer Chemistry. Pearson Education Inc. Upper Saddle River, N.J. p. 523.
37. Yang, C., Xing, J., Guan, Y. Liu, H. 2006. Superparamagnetic poly(methyl methacrylate) beads for nattokinase purification from fermentation broth. Appl. Micro. And Biotech. Vol. 72 No. 3, 616-622.
38. Stuart M K, Chamberlain N R. 2003. Monoclonal antibodies to elongation factor-1alpha inhibit in vitro translation in lysates of Sf21 cells. Arch Insect Biochem Physiol. Vol. 52, No. 1, 17-34
39. Ioannou Y, Giles I, Lambrianides A, Richardson C, Pearl L H, Latchman D S, Isenberg D A, Rahman A. 2006. A novel expression system of domain I of human beta2 glycoprotein I in Escherichia coli. BMC Biotechnol. Vol. 6, 8.
40. Hattori, T., Halberg, R., Dubin, P L. 2000. Roles of Electrostatic Interaction and Polymer Structure in the Binding of Beta-Lactoglobulin to Anionic Polyelectrolytes: Measurement of Binding Constants by Frontal Analysis Continuous Capillary Electrophoresis. Langmuir, Vol. 16 No. 25, 9738-9743.
41. Allcock, H R., Lampe, F W, Mark, J E. 2003. Contemporary Polymer Chemistry. Pearson Education Inc. Upper Saddle River, N.J. pp. 632-634.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

What is claimed is:

1. An antigen-capture substrate comprising
a solid surface coated with a polymer; and
an antibody-binding protein coupled to the polymer, wherein the antibody-binding protein can bind an Fc region of an antibody, and wherein the polymer is poly-methyl methacrylate (PMMA), poly-acrylic acid, poly-styrenesulfonate/poly-2,3-dihydrothieno(3,4-b)-dioxin, poly-aniline or poly-styrene-co-methyl methacrylate.

2. The substrate of claim 1, wherein the polymer is poly-methyl methacrylate (PMMA).

3. The substrate of claim 1, wherein the antibody-binding protein is protein G.

4. The substrate of claim 1, wherein the antibody-binding protein is a truncated protein G.

5. The substrate of claim 1, wherein the antibody-binding protein is protein G′ expressed in Streptococci sp. G148.

6. The substrate of claim 1, wherein the antibody-binding protein is protein A.

7. The substrate of claim 1, wherein the antibody-binding protein is protein L.

8. The substrate of claim 1, further comprising an antibody bound to the antibody-binding protein.

9. The substrate of claim 8, wherein the antibody is an IgG.

10. The substrate of claim 9, wherein the IgG is a human IgG.

11. The substrate of claim 1, wherein the solid surface is polystyrene.

12. The substrate of claim 1, wherein the solid surface is a multi-well plate.

13. The substrate of claim 1, wherein the solid surface is glass.

14. The substrate of claim 1, wherein the solid surface is a slide.

15. The substrate of claim 1, wherein the antibody-binding protein is non-covalently coupled to the polymer.

16. A method for producing an antigen-capture substrate, the method comprising:
(a) coating a solid surface with a polymer; and
(b) coupling an antibody-binding protein to the polymer, wherein the antibody-binding protein can bind an Fc region of an antibody, and wherein the polymer is poly-methyl methacrylate (PMMA), poly-acrylic acid, poly-styrenesulfonate/poly-2,3-dihydrothieno(3,4-b)-dioxin, poly-aniline or poly-styrene-co-methyl methacrylate.

17-22. (canceled)

23. The method of claim 16, further comprising coupling an antibody to the antibody-binding protein.

24. The method of claim 23, wherein the antibody is an IgG.

25. (canceled)

26. (canceled)

27. The method of claim 16, wherein the solid surface is a multi-well plate.

28-32. (canceled)

33. A method for determining presence of an antigen in a sample, the method comprising
(a) contacting the antigen-capture substrate of claim 8 with the sample, wherein the antibody binds specifically to the antigen when the antigen is present; and
(b) determining if the antibody bound the antigen,
wherein if the antibody bound the antigen, the antigen is determined to be present in the sample.

34-38. (canceled)
